(19) United States
(12) Patent Application Publication
(10) Pub. No.: US 2023/0145832 A1
(43) Pub. Date: May 11, 2023

(54) MEDICAL TREATMENTS BY COGNITIVE DISCONNECTION

(71) Applicants: The Cleveland Clinic Foundation, Cleveland, OH (US); The United States Government as Represented by the Department of Veterans Affairs, Washington, DC (US)

(72) Inventors: Paul D. Marasco, Lakewood, OH (US); Jonathon S. Schofield, Davis, CA (US); Courtney E. Shell, Round Rock, TX (US); Zachary C. Thumser, Cleveland, OH (US); Dylan Beckler, Lyndhurst, OH (US); Jonathan Sakai, Rocky River, OH (US)

(21) Appl. No.: 17/980,164

(22) Filed: Nov. 3, 2022

Related U.S. Application Data
(60) Provisional application No. 63/276,068, filed on Nov. 5, 2021.

(51) Int. Cl. A61M 21/02; A61M 5/52

(52) U.S. Cl. CPC A61M 21/02; A61M 5/52; A61M 2021/0022

(57) ABSTRACT

A cognitive illusion of ownership over a proxy extremity (e.g. a hand) is provided by discordant touch and visual information to a user. While the user feels the touch on their real extremity, they see a corresponding touch on the proxy extremity. The user's internally modeled reality is then updated to move the felt location of the real extremity to the seen location of the proxy extremity. As a result, the user embodies the proxy extremity, thereby disembodying the real extremity. Pain associated with medical procedures performed on the real extremity is then mitigated. And because an epileptic brain is less stringent when resolving conflict between what is seen and what is felt, correlations in time or space can shift attribution of the sensation of the felt touch on the user's real extremity to the location of the proxy extremity. This can be helpful in treating and/or diagnosing epilepsy.

### CROSS-REFERENCE TO RELATED APPLICATIONS

This application claims priority to U.S. Provisional Patent Application No. 63/276,068 filed on Nov. 5, 2021, the entirety of which is incorporated herein by reference.

### STATEMENT REGARDING FEDERALLY SPONSORED RESEARCH OR DEVELOPMENT

This invention was made with government support under NS081710 awarded by the National Institutes of Health and the Department of Defense (DARPA) P-1108-114403 and (CDMMRP) W81XWH-15-1-0575. The government has certain rights in the invention.

### BACKGROUND

Needle procedures (such as injections, IV placements, and venipunctures) are some of the most frequently performed clinical interventions, with blood draws occurring in nearly 40% of emergency department visits. Particularly for children, needle sticks induce pain in nearly 76% of pediatric patients and are described by children as one of the most traumatic aspects of visits to the hospital and doctor. Thus, these procedures can be painful and distressing, and lead to anxiety, loss of sleep, and resistance to medical visits. In view of this, effective needle pain management is an important driver of whether a patient will receive adequate healthcare as a child and throughout their adult life. Indeed, needle pain and the accompanying distress are well-documented contributors to healthcare avoidance behaviors and vaccine refusal in children and their guardians, as well as later in life when the child becomes an adult. The efficacy of conventional needle pain management strategies, which may include topical anesthesia, tactile stimulation, and conversational distraction techniques, remains debated and the challenges surrounding needle pain are still a pervasive clinical issue.

Separately, seizure disorders (e.g., epilepsy) chronically affect tens of millions of people. Individual seizures are caused by abnormally neuronal activity in the brain, and can be evidenced by decreased consciousness, automatism, tonic-clonic convulsions, and the like. Because of these outward symptoms, seizure disorders can affect an individual's ability to work and perform daily functions, and can be stigmatized.

### BRIEF SUMMARY

According to one example of the present disclosure a method comprises: operating a first sensory actuator with respect to an extremity of a user; operating a second sensory actuator with respect to a proxy extremity, the proxy extremity corresponding to the extremity of the user; and performing a medical procedure on the extremity of the user, wherein operation of the first sensory actuator is visually hidden from the user and induces a non-visual sensory response in the extremity of the user, and wherein operation of the second sensory actuator is visible to the user and does not induce the non-visual sensory response in the user.

In various embodiments of the above example, the extremity is a finger or hand; the extremity is a toe or foot; the first sensory actuator and the second sensory actuator forming a sensory actuator pair and the method comprising operating a plurality of sensory actuator pairs, wherein each sensory actuator pair corresponds to a different extremity of the user, and wherein the sensory actuators of each sensory actuator pair are operated simultaneously; the first sensory actuator and the second sensory actuator forming a sensory actuator pair and the method comprising operating a plurality of sensory actuator pairs, wherein each sensory actuator pair corresponds to a different extremity of the user, and wherein the sensory actuators of each sensory actuator pair are operated asynchronously; the first sensory actuator and the second sensory actuator are operated until the user at least partially disembodies the extremity; the first sensory actuator is not visible to the user during operation of the first and second sensory actuators, and the second sensory actuator is visible to the user during operation of the first and second sensory actuators; and/or the first sensory actuator is a rack and pinion actuator, and operating the first sensory actuator comprises controlling a servo motor of the rack and pinion.

According to another example of the present disclosure, a system comprises: a lower frame; an upper frame rotatably attached to the lower frame; a real hand plate attached to the lower frame and configured to hold a hand of a user; a proxy hand plate attached to the upper frame; and a proxy hand held on the proxy hand plate, wherein the real hand plate comprises a plurality of first sensory actuators and the proxy hand plate comprises a plurality of second sensory actuators, and wherein each one of the second sensory actuators is at a location of the proxy hand corresponding to a location of one of the first sensory actuators of the user's hand.

In various embodiments of the above example, each of the first sensory actuators is at a different finger of the user's hand, and each of the second sensory actuators is at a different finger of the proxy hand; the real hand plate and the proxy hand plate each comprise a plurality of finger clamps attached to the plate, each of the plurality of finger plates housing a different one of the first and second sensory actuators and being configured to hold a finger of the user's hand or the proxy hand; each of the first and second sensory actuators is a rack and pinion actuator comprising a servo configured to rotate the pinion; the system further comprises a processor configured to: simultaneously operate one of the first sensory actuators and the one of the second sensory actuators at the corresponding location of the proxy hand; the system further comprises a processor configured to: operate one of the first sensory actuators and the one of the second sensory actuators at the corresponding location of the proxy hand at different times, or simultaneously operate one of the first sensory actuators at a location of the hand of the user and one of the second sensory actuators at a different location of the proxy hand; an angle between the lower frame and the upper frame is greater than zero degrees such that when the sensory actuators are operated, the user's hand and the first sensory actuators are not visible to the user, and the proxy hand and the second sensory actuators are visible to the user; and/or the proxy hand is a digital representation of the hand of the user on a display, the display being held on the proxy hand plate.

According to still another example of the present disclosure, a system comprises: a touch feedback device comprising a plurality of first sensory actuators and configured to hold an extremity of a user; a proxy extremity; and a processor configured to operate one of the first sensory actuators a location of the extremity of the user corresponding to an identified location of the proxy extremity, wherein the extremity of the user and the proxy extremity are in a visually separated fields of view, and wherein the plurality of first sensory actuators are configured to induce a sensory response of the user when operated.

In various embodiments of the above example, the system further comprises: a wand, wherein the processor is configured to determine the identified location of the proxy extremity based on a proximity or touch of the wand to the proxy extremity; the processor is further configured to: predict the identified location of the proxy extremity based on a movement of the want or the proximity of the wand to the proxy extremity, and operate the one of the first sensory actuators prior to the wand touching the proxy extremity; and/or the proxy extremity is a digital representation of the extremity of the user.

### BRIEF DESCRIPTION OF THE SEVERAL VIEWS OF THE DRAWING

FIG. 1 is a model of the relationship between a person's internally modeled reality, an externally applied reality (e.g., an illusory reality), and actual reality.

FIG. 2 illustrates example disembodiment device according to the present disclosure.

FIG. 3 illustrates a top view of the lower frame of the example disembodiment device of FIG. 2.

FIGS. 4A and 4B illustrate top and bottom views, respectively, of the upper frame of the example disembodiment device of FIG. 2.

FIGS. 5A and 5B illustrate hand plates for real and proxy hands, respectively, of the example disembodiment device of FIG. 2.

FIGS. 6A, 6B, and 6C illustrate front, side, and perspective views of an example finger clip of the hand plates of the example disembodiment device of FIG. 2.

FIG. 7 illustrates an example application of the example disembodiment device of FIG. 2.

FIG. 8 illustrates a folded carriable configuration of the example disembodiment device of FIG. 2.

FIG. 9 illustrates a model of the relationship of individual modeled realities of two agents cooperating in a shared actual reality.

FIG. 10 illustrates an additional example cognitive illusion system according to the present disclosure.

### DETAILED DESCRIPTION

The systems and methods of the present disclosure induce a ‘cognitive/perceptual illusion’ that can be used to reduce pain during needle procedures and diagnose and treat seizure disorders, such as epilepsy. The systems and methods are also applicable to other outpatient procedures including, but not limited to, grafts, minor excisions, trauma/wound treatments, stitching, and the like, which can cause pain to the user.

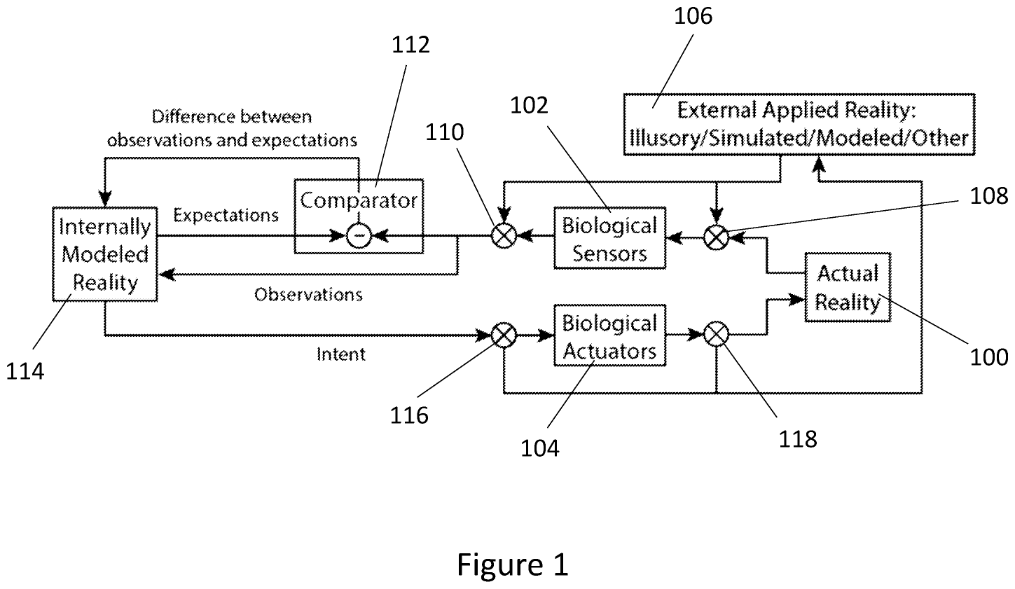

The cognitive illusion is based on the relationship between a person's internally modeled reality, an externally applied reality (e.g., an illusory reality), and the actual reality. This relationship is modeled in FIG. 1. Therein, the actual reality 100 relates to a state of actual reality that an individual samples by way of their biological sensors 102, and interacts with by way of their biological actuators 104. The biological sensors 102 may be, for example, the individual's sensory receptors such as for touch, taste, sight, smell, and sound; and the biological actuators 104 may be, for example, those related to motion control such as movement of the individual's arms, hands, legs, and feet (e.g., controlled by muscle action).

The reality sampled by biological sensors 102 may be modified by an externally applied reality 106, where valve 108 represents the combination of the actual reality 100 and the externally applied reality 106. The actual reality 100 combined with the externally applied reality 106 combined at valve 108 are together sampled by the biological sensors 102. Modifications of the sampled reality can include, but are not limited to, inputs such as tactile, visual, auditory, olfactory, thermal, nociceptive, proprioceptive, and gustatory either alone or in relation to anticipated outcomes.

The output of the biological sensors 102 can again be modified by the externally applied reality 106 (at valve 110) before being sent through the individual's nervous system to comparator 112. Modifications through valve 110 can include, but are not limited to, stimulatory approaches such as electrical, magnetic, chemical, mechanical, illusory, ultrasound, optical, thermal, anesthetic, and nerve redirection. In other words, these modifications are not those that would be sampled by biological sensors 102. Rather, the modifications introduced at valve 110 are to the outputs of the biological sensors 102 themselves (e.g., to the electrophysiological signals generated by the sensors 102).

The internally modeled reality 114 then is the individual's ideas, assumptions, predictions, and the like about the individual's place. This internally modeled reality 114 is created from the senses, observations, and learned experiences, and provides the individual with a framework by which to anticipate, predict, expect, and/or modulate their engagement with their reality.

The comparator 112 continuously monitors sensory information for discrepancies from internally modeled expectations 114. Resulting differences between observations (from biological sensors 102) and expectations (of the internally modeled reality 114) are used to update the internally modeled reality 114. In other words, the internally modeled reality 114 is composed of the differences between observations and expectations arising from the comparator 112 and generates and/or modifies expectations or intent.

The output of the individual's internally modeled reality 114 is supplied to the biological actuators 104, as a person intends to interact with the actual reality 100. The output of the internally modeled reality 114 may be combined with (and thus modify) the externally applied reality 106 at valve 116, prior to being supplied through the individual's nervous system to the biological actuators 104. Similar to internal modifications of the biological sensors at valve 110, modifications through valve 116 can include, but are not limited to, stimulatory approaches such as electrical, magnetic, chemical, mechanical, illusory, ultrasound, optical, thermal, anesthetic, and nerve redirection.

Similarly, the output of biological actuators 104 may modify the externally applied reality 106 at valve 118. Such modifications can alter the individual's movements, and change physical outcomes. These changes affect the actual reality 100, which is then detected by biological sensors 102 as described above.

As this relates to pain induced by medical procedures (e.g., needle procedures), the model of FIG. 1 can be exploited by generating a cognitive illusion of ownership over a proxy extremity by providing discordant touch and visual information. It should be understood that while the present disclosure generally relates to procedures on a hand, it should be understood that the systems and methods of the present disclosure are applicable to any extremity or limb, such as fingers, hands, toes, feet, legs, arms, and the like. Further, it should be understood that the proxy extremity may be a prosthetic, digital representation (e.g., on a tablet or like display screen), or the like.

In generating the cognitive illusion of ownership over a proxy hand, a user feels a touch on their real hand, but sees the touch occurring on the proxy hand. This mismatch enters the model of FIG. 1 at valve 108, where the biological sensors 102 in the fingertips feel touch in one place (the user's real hand), and the biological sensors 102 of the eyes see touch occurring in a different place (the proxy hand). The comparator 112 then registers the mismatch between the seen touch and the felt touch, and the internally modeled reality 114 is updated to move the felt location of the real hand to the seen location of the proxy hand. This results in the user embodying the proxy hand, thereby disembodying (cognitively neglecting) the real hand. With the user's real hand disembodied, pain and other negative sensations caused by procedures performed on the real hand can be mitigated.

According to some embodiments, the user's hand is placed into a disembodiment device that holds both the user's hand and fingertips, and the hand and fingertips of a proxy hand. More particularly, the user's hand remains flat on a table with the palm up, while the proxy hand is also arranged palm up and oriented at an angle such that the proxy hand is visible to the user while obscuring the real hand. In this way, the proxy hand represents an externally applied reality 106 that replaces the user's real hand as detected by the biological sensors 102 (the user's eyes). In other words, as detected by the biological sensors 102, the actual reality 100 (the user's real hand) is modified by the externally applied reality (the proxy hand) at valve 108.

The disembodiment device has a first set of sensory actuators associated with the real hand, and a second set of sensory actuators associated with the proxy hand. The first set of sensory actuators is operated to cause a tactile sensory response on the user's real hand, but cannot be seen by the user. Conversely, the second set of sensory actuators is operated to be seen by the user to be engaging with the proxy hand, but does not cause a tactile sensory response. In other words, operation of the sensory actuators results in activation of the biological sensors 102 in the real hand (e.g., to produce a haptic feeling). In this regard, the sensory actuators may take any form that would cause a sensory response. For example, each sensory actuator may be a device that directly touches the real hand, or one that results in the sensation of a touch (e.g., by releasing pressurized air, or increasing air pressure at the hand, or the like).

Again operating at valve 108 to modify the actual reality 100, the first set of sensory actuators is synchronously (at the same time and relative place on the corresponding hand) operated with the second set of sensory actuators in the case of reducing pain during a medical procedure (e.g., needle procedures). The timing and location of sensory actuator operation may be random and last on the order of a few seconds to a few minutes. This induces a sensory mismatch between the visual and tactile sensory information streams from biological sensors 102 that are read and compared at the user's brain (the comparator 112). The sensory mismatch between what is seen and what is felt induces conflict in the user's internally modeled reality 114.

To resolve the conflict between what is seen and what is felt, the expectations generated by the internally modeled reality 114 are realigned with respect to the observed sensory mismatch to minimize the discrepancy identified by comparator 112. This cognitive realignment of the user's internally modeled reality 114 shifts attribution of the sensation of the felt touch on the user's real hand to the location of the proxy hand that is in view. The realignment of felt touch to the proxy hand causes the cognitive illusion that the proxy hand is part of the user's body.

Simultaneously, the realignment of felt touch to the proxy hand leads to cognitive neglect of the user's real hand and disembodiment of the user's real hand from their body image. As a result, pain felt by a needle (or other procedure) in the user's real hand is reduced.

And as this relates to the diagnosis and treatment of seizure disorders such as epilepsy, a comparator 112 of an epileptic brain is less stringent when resolving conflict between what is seen and what is felt. In this case the requirement of specific context related to the simultaneous presentation of both seen and felt sensations for cognitive realignment is lost. As such, simple correlations in time (such as seen touches on the proxy hand that follow felt touches to the real hand by a brief yet equidistant interval) or space (such as seen touches on a digit of the proxy hand that are simultaneously felt on a different digit of the real hand) results in maladaptive cognitive realignment of the user's internally modelled reality 114, which shifts attribution of the sensation of the felt touch on the user's real hand to the location of the proxy hand in view.

In the case of diagnosing and/or treating epilepsy, the first set of actuators is asynchronously operated with the second set of actuators. Particularly, operation of the first and second sets of actuators is offset either temporally or spatially. The shift to attribution of the sensation of the felt touch on the user's real hand to the location of the proxy hand in view under temporal or spatial mismatch thus reveals the maladaptive propensity for updating the user's internally modelled reality 114 based on non-contextual simple visual and temporal correlations.

Considering this, when diagnosing and/or treating epilepsy, the first and second sets of sensory actuators would not operate synchronously as described above. Rather, the sensory actuators are operated asynchronously, where operation of a pair of sensory actuators (e.g., one of the first set of sensory actuators and one of the second set of sensory actuators) are separated in time or space. For example, a pair of sensory actuators associated with the same digit are operated at different times. Alternatively, a pair of sensory actuators associated with different digits are operated at the same time. Because spatial distance can affect the comparator 112 of the epileptic brain, the digits associated with a pair of sensory actuators operated at the same time may be selected based on their relative locations.

In one example embodiment, digits D1-D3 (thumb, index, and middle, respectively) may be characterized as a first group and digits D4-D5 (ring and little/pinky, respectively) may be characterized as a second group. In such an embodiment, the sensory actuator of each pair may be associated with a digit in different groups. For example, the sensory actuator from the first set of sensory actuators may be associated with digit D2 and the sensory actuator from the second set of sensory actuators may be associated with digit D4. In this case, the sensory actuator associated with user's real digit D2 is operated at the same time the sensory actuator associated with the proxy digit D4 is operated.

According to another example embodiment, a first digit is selected (e.g., by random) and a second digit is then determined as the digit that is farthest away. For example, if digit D1 is selected on the user's real hand, then digit D5 is selected on the proxy hand. Thus, the sensory actuator associated with digit D1 of the first set of sensory actuators is operated at the same time the sensory actuator associated with digit D5 of the second set of sensory actuators is operated. In still another embodiment, there is at least one digit between the digits associated with the sensory actuator pair. In other words, sensory actuators are not operated at the same time for the same or adjacent digits. For example, the sensory actuator pairs may be associated with digits D1 and D3, D1 and D4, D1 and D5, D2 and D4, D2 and D5, or D3 and D5.

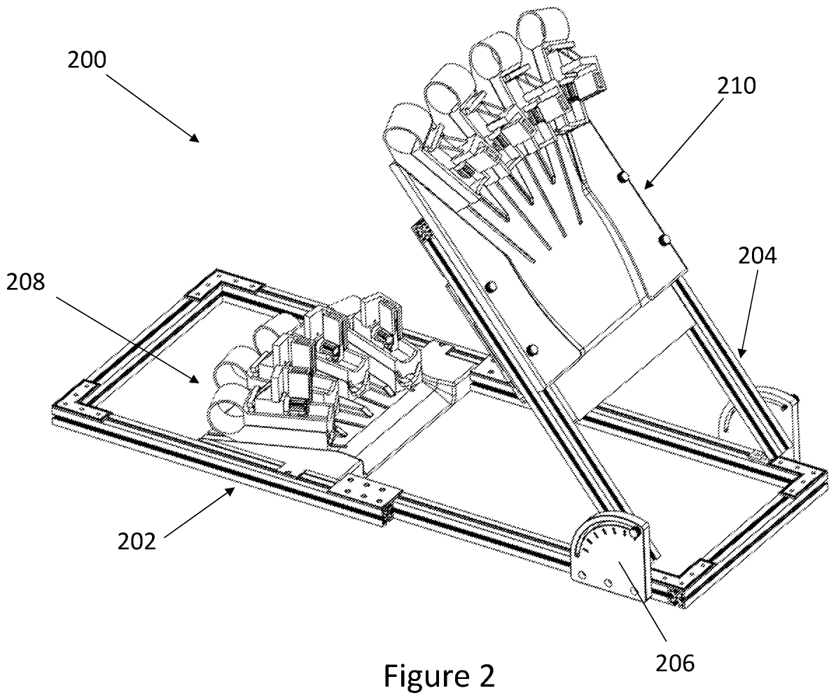

An example disembodiment device 200 according to the above embodiments is illustrated in FIG. 2. The device 200 has a lower frame 202 and an upper frame 204 rotatably mounted to each other at one end by a hinge 206. As seen in the figure, the upper frame 204 may be rotatable to about 90 degrees with respect to the lower frame 202. Rotation of the upper frame 204 relative to the lower frame 202 may be manual, or automated/electrically controlled. A hand plate 208 that supports and holds the user's real hand is attached to the lower frame 202; and similarly, a hand plate 210 that supports and holds the proxy hand is attached to a top side of upper frame 204. In embodiments where the proxy hand is digitally represented, the upper frame 204 may hold a tablet or like display screen that displays the digital representation of the proxy hand. An electronics enclosure (not visible) for operating the disembodiment device 200 is attached to a bottom side of upper frame 204.

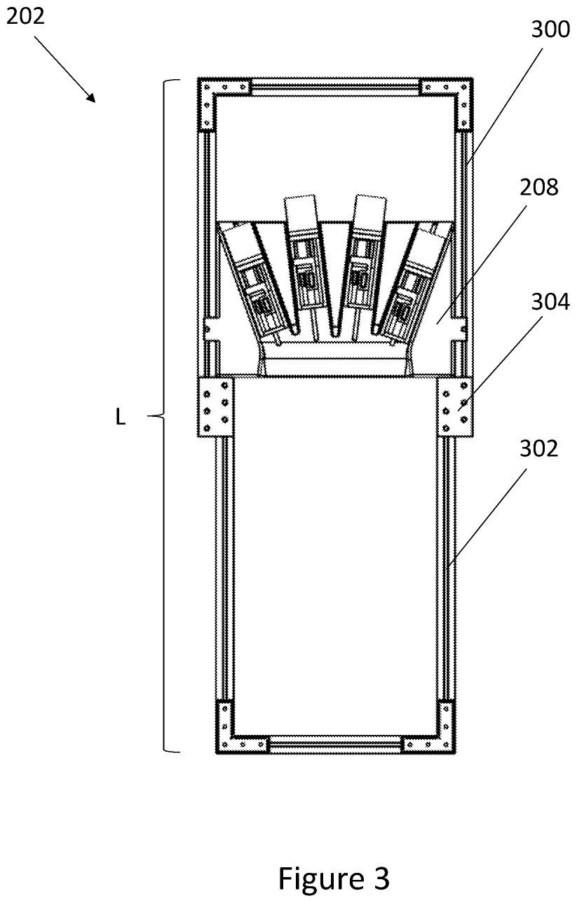

FIG. 3 illustrates a top view of the lower frame 202. As seen therein, the lower frame 202 includes an upper frame portion 300 and a lower frame portion 302. Each frame portion 300, 302 is substantially square or rectangular with three sides (thus having an open end). The open ends of the frame portions 300, 302 are connected to each other by brackets 304. The hand plate 208 for the real hand is connected to the upper frame portion 300.

The frame portions 300, 302 preferably have tracks (e.g., extrusions, cutouts, grooves, rails, or the like) to which the hand plate 208 and brackets 304 can be attached. Accordingly, the attachment points of the hand plate 208 and brackets 304 to each portion 300, 302 can be adjusted along to any point of the frame portions 300, 302 to suitably accommodate a user's hand and arm. In other words, a total length L of the lower frame 202, and relative position of hand plate 208, can be adjusted according to a user's arm and hand length/size.

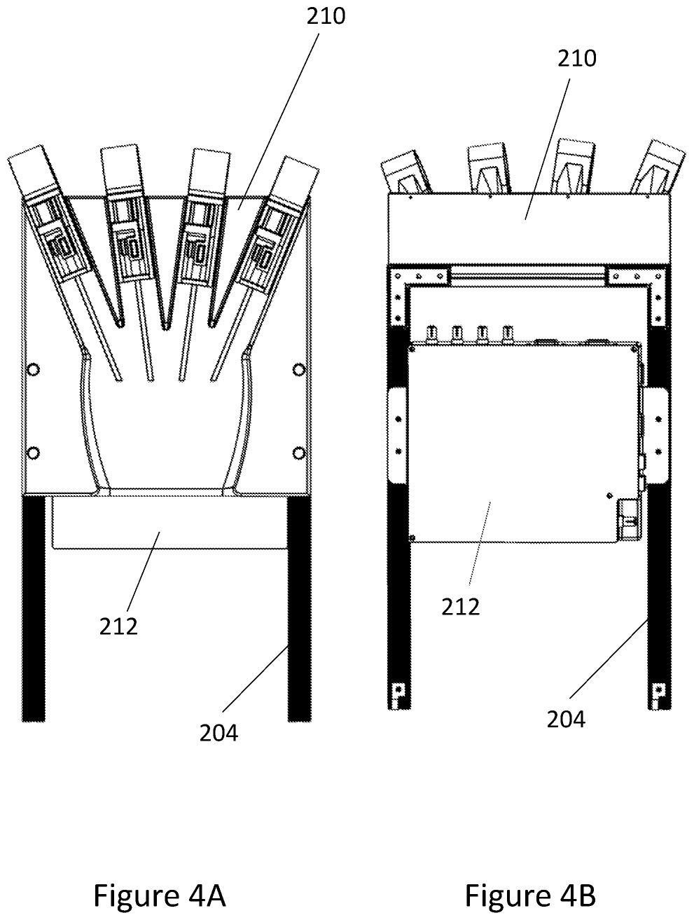

FIGS. 4A and 4B illustrate top and bottom views, respectively, of the upper frame 204. Similar to the lower frame 202, hand plate 210 is attached to a top side of the upper frame 204. The electronics enclosure 212 is attached to a bottom side of the upper frame 204. As with the connections to lower frame 202, the upper frame may also include tracks to which the hand plate 210 and electronics enclosure 212 may be attached, so that their locations are adjustable.

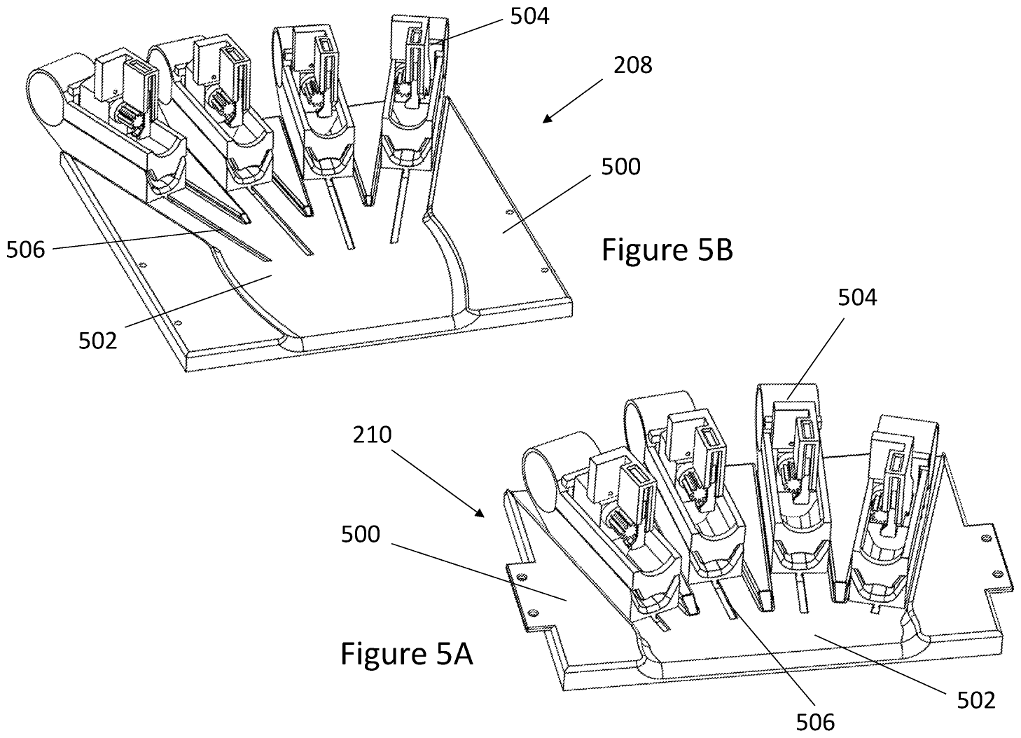

FIGS. 5A and 5B further illustrate hand plates 210 and 208, respectively, in more detail. Each hand plate includes a base 500, which is attached to the corresponding frame 202, 204. The base 500 may include a cutout 502, extrusion, or the like that substantially corresponds to the shape of the hand. The cutout 502 helps properly align and secure the user's real hand (or the proxy hand) snuggly with the base 500.

The hand plates 208, 210 further include finger clips 504 attached to the plates 208, 210 in a portion of the cutout 502 corresponding to at least one finger. Although the example of FIGS. 5A and 5B illustrate four finger clips 504 (one associated with each finger other than thumb), any number may be utilized. The finger clips 504 further secure the user's real hand or the proxy hand to the corresponding base 500, and house the sensory actuators that touch the hand. The finger clips 504 are attached to the base 500 in tracks 506. In this manner, the length of the finger can be adjusted to fit a user's hand size by moving finger clips 504 along the track.

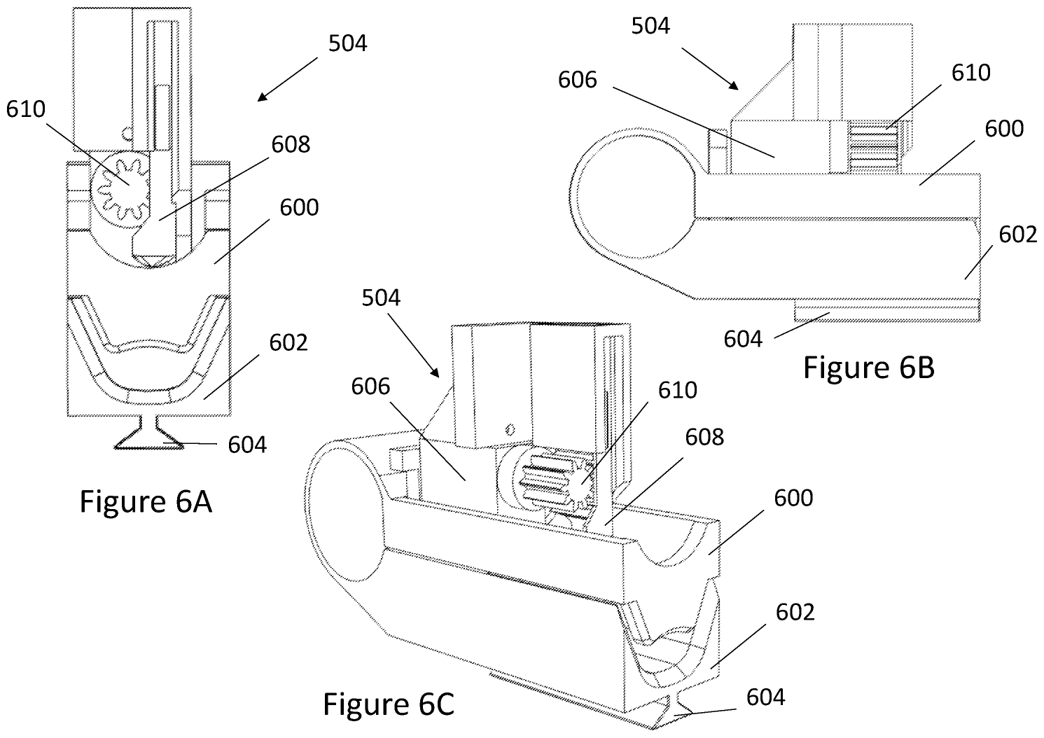

The finger clips 504 are illustrated in more detail in FIGS. 6A-6C, which show front, side, and perspective views, respectively. The finger clips 504 include an upper portion 600 and lower portion 602, which are configured to hold a finger therebetween. For example, the upper portion 600 and lower portion 602 may be spring-loaded or otherwise biased in a closed state so as to pinch the finger from the top and bottom. A dove-tail 604 or like element underneath the lower portion 602 can engage with the tracks 506 of the plates 208, 210. In other words, the finger clips 504 may be attached to the plates 208, 210 with a sliding dovetail joint.

The upper portion 600 of the finger clip 504 may include a slot or like aperture through which the sensory actuator may engage with the finger. In the example of FIGS. 6A-6C, the sensory actuator is of a plunger-type and includes a servo motor 606, which drives a rack 608 and pinion 610. The servo motor 606 drives rotation of the pinion 610, which causes the rack 608 to move up and down (plunge) through the aperture in the upper portion 600 of the finger clip 504. While a rack and pinion actuator is described herein, it should be understood that any actuator may be utilized. For example, the sensory actuator may be of a piston-type actuator in which hydraulic, pneumatic, or an electrical motor cause a piston to extend from a cylinder towards the finger. In some examples, one or more of the sensory actuators may be mechanically linked for example by a cam shaft causing synchronized motion of the pistons. In still other examples, the sensory actuator may release pressurized air (e.g., by opening a valve), or otherwise cause a burst of pressurized air, toward the finger. In these examples, the sensory actuator itself does not directly engage the finger. Preferably the sensory actuator is visible to the user so that they may see when and where actuation occurs.

Operation of the disembodiment device 200 may be wholly or partially controlled by electronics in the electronics enclosure 212. The electronics enclosure 212 may house at least a processor, memory, and power source. The power source may be an internally housed battery or a power supply connectable to a mains power (e.g., a wall outlet). The processor is configured to output control signals to each of the sensory actuators of the device 200. These control signals may be supplied to the sensory actuators, for example, via communication cables removably attached to the electronics enclosure 212 at one end, and to the sensory actuators of the corresponding hand plates 208, 210 at another end, via communication ports at the electronics enclosure 212 and the individual sensory actuators or the plates 208, 210.

The processor may further be configured to communicate with external or otherwise remote devices through wired or wireless connections. For example, the processor may be further configured to control operation of lights associated with each sensory actuator. Control signals for the lights may then be supplied from the electronics enclosure 212 via expansion ports of the electronics enclosure. In other examples, the processor may be configured to communicate with a remote control processor (e.g., a remote server) via a wired connection or wireless network. In this manner, the disembodiment device 200 may be remotely operated or monitored.

The electronics enclosure 212 may further include human-machine-interface (HMI) elements. For example, power switches may control the supply of power to the processor (and thus the whole device) and/or the hand plates 208, 210 individually. Further, the position of each sensory actuator may be adjusted by knobs or the like, for example, by adjusting potentiometers. The sensory actuator position adjustment may allow for accommodation of different finger diameters and placements within the finger clips 504. Furthermore, the sensory actuator position adjustment may allow for close proximity placement of the sensory actuator to the finger. The processor may be configured to adjust control of each sensory actuator (e.g., a power, speed, magnitude, or the like). Still further, a mode selection switch(es) may be used to select an operation program executed by the processor. Such programs may control the sequence, order, timing, and like operation of each of the sensory actuators. Changing the sequence, order, and timing of actuation may allow for modifications to the experience of the ownership illusion, and adjusting experimental controls and for exploring comparator function during research. For example, such modes can include a needle stick (or like) procedure mode in which the sensory actuators of each plate 208, 210 are operated synchronously, an epilepsy diagnostic or treatment mode in which the sensory actuators of each plate 208, 210 are operated asynchronously, and/or a research mode in which the sensory actuators of each plate 208, 210 can be specially controlled according to a research protocol.

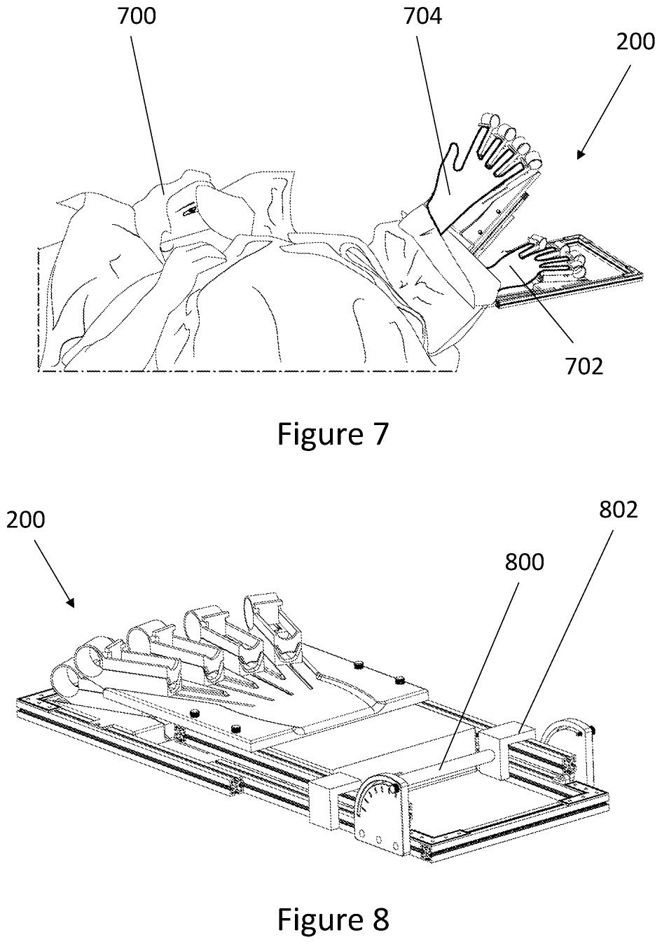

With reference to FIG. 7, the disembodiment device 200 may be employed for example in a surgical setting. In such an environment, a user 700 is on their back, and their real hand 702 is held to the hand plate 208 of the lower frame 202 of the disembodiment device 200, with their four fingers (which may include or exclude the thumb) held in the corresponding finger clips 504. A proxy hand 704 (and the fingers thereof) is similarly held in plate 210 of the upper frame 204. Preferably the skin tone of the proxy hand 704 substantially matches that of the user's real hand 702 to further strengthen the visual illusion of the proxy hand. The upper frame 204 is raised at approximately a 60 degree angle with respect to the lower frame 202.

In the configuration of FIG. 7, only the proxy hand 704 is visible to the user 700. The processor of the disembodiment device 200 may control operation of each sensory actuator according to a program, for example, so that sensory actuators of corresponding fingers of the real hand 702 and proxy hand 704 are synchronously operated. As discussed above, this induces the cognitive illusion that causes the user to embody the proxy hand 704 and disembody the real hand 702.

When not in use, the disembodiment device 200 may be folded and carriable. As illustrated in FIG. 8, the upper frame 204 is rotated such that the angle between the upper frame 204 and lower frame 202 approaches zero degrees. In other words, the upper frame 204 may be folded down so that it essentially rests on top of the lower frame 202. A carry bar 800 spanning a width of the disembodiment device may be attached to the upper and lower frames 202, 204 by clips 802. In addition to attaching the carry bar 800 to the device 200, the clips 802 further serve to constrain rotation of the upper frame 204 and prevent the device 200 from opening. The carry bar 800 may be used as a handle to carry or otherwise transport the device 200.

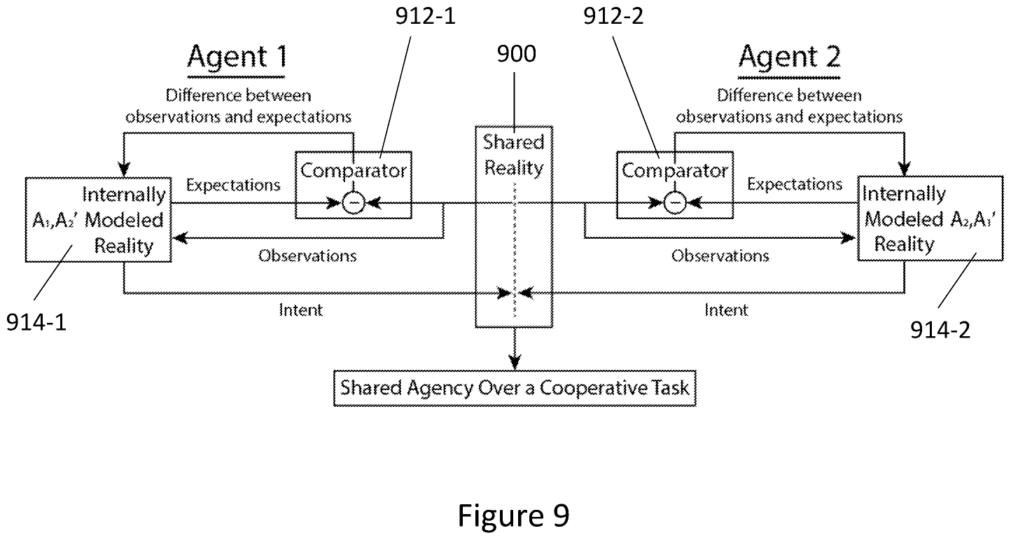

Turning now to FIG. 9, the model of FIG. 1 may be reimagined for two cooperative agents. As used herein, an agent may be a human, intelligent machine, HMI, or the like that can sense and/or interact with an actual reality. In essence, FIG. 9 represents two agents, each modeled according to FIG. 1, but sharing a common actual reality. More particularly, as seen in FIG. 9, each agent (Agent 1, Agent 2) shares a common actual reality 900. This shared reality 900 is analogous to the actual reality 100 of FIG. 1, which is detectable by each agent's sensors (e.g., biological sensors 102 in the case of a human agent, or transducer such as a touch sensor, light sensor, camera, microphone, or the like in the case of an intelligent machine or HMI).

The sensed shared reality of each agent is then compared at comparator 912-1, 912-2 (analogous to comparator 112 of FIG. 1) with the agent's expectations based on their internally modeled reality 914-1, 914-2 (analogous to the internally modeled reality 114 of FIG. 1). Each agent may then interact with and affect the shared reality 900 with their actuators (e.g., biological actuators 104 in the case of a human agent, or electrical or mechanical actuators such as displays, motors, speakers, or the like in the case of an intelligent machine or HMI) based on their internally modeled reality 914-1, 914-2.

Because both agents interact with the same shared reality 900, the internally modeled reality for each agent is affected by the internally modeled reality of the other agent. For example, the internally modeled reality 914-1 of Agent 1 corresponds to shared reality 900 as modified by Agent 2 (based on the internally modeled reality 914-2 of Agent 2) and sensed by Agent 1. In this sense, action of each agent on the shared reality has a similar effect to the externally applied reality 106 of FIG. 1.

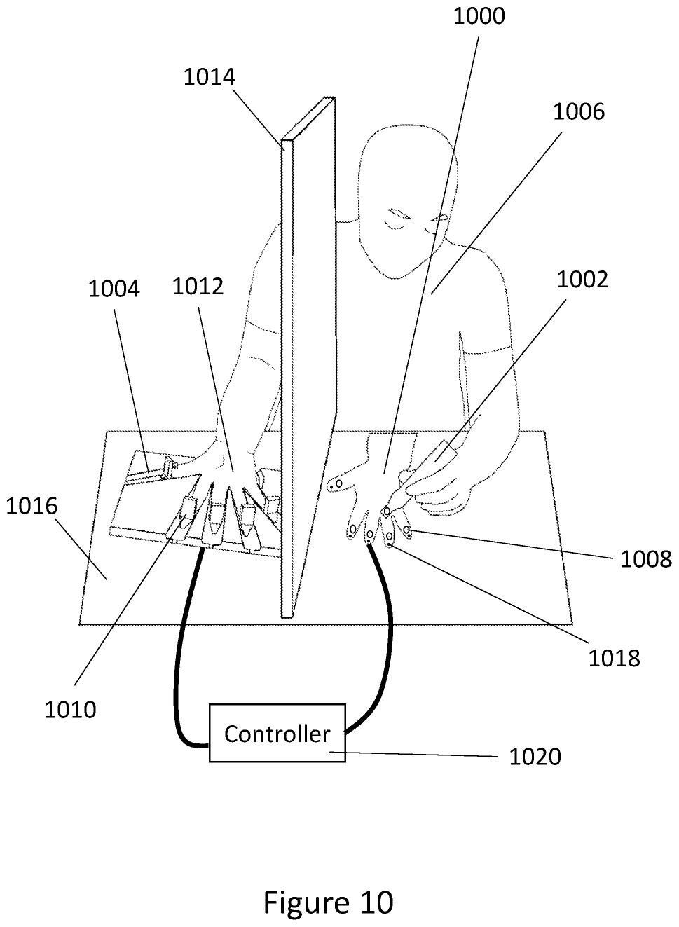

Based on the model of FIG. 9, other embodiments of the present disclosure may realize the cognitive illusion described herein according to the example of FIG. 10. More particularly, the example of FIG. 10 utilizes a sensorized proxy (e.g., rubber) limb 1000, a touch device (e.g., a wand) 1002, a controller (e.g., a processor, computer, discrete controller, or the like) 1020, and a touch feedback device 1004 (collectively corresponding to Agent 2 of FIG. 9). The sensorized proxy limb 1000 may also be embodied in a digital/electronic form such as the display of an animated artificial hand on a screen such as a tablet device or smartphone.

In operation, a user 1006 (corresponding to Agent 1 of FIG. 9) uses the wand 1002 to touch the proxy limb 1000. The proxy limb 1000 and/or the wand 1002 may include sensors 1008 such as touch, pressure, capacitive, proximity sensors (e.g., infrared), accelerometers, and/or the like in the tip of the wand 1002 and at the fingertips or other locations of the proxy limb 1000. For example, in some embodiments, the proxy limb 1000 may have proximity or touch sensors to detect an actual touch or proximity of the wand 1002 on the proxy to preemptively infer an anticipated touch. Alternatively or additionally, the wand 1002 may include a pressure sensor to detect the strength of the touch and/or accelerometers to determine movement characteristics of the wand 1002 as it is controlled by the user to preemptively infer the anticipated touch. In short, the sensors 1008 are configured to detect the touch and/or presence of a wand 1002 controlled by the user 1006 with a non-procedure hand.

In some embodiments such as that in FIG. 10, the touch feedback device 1004 may be similar to the hand plate 208 for user's real hand discussed above. For example, the touch feedback device 1004 may include sensory actuators 1010 (e.g., vibration devices, servo motors, piezoelectric elements, and the like) at locations corresponding to the sensors in the proxy hand (e.g., at the finger tips). Each sensor 1008 of the proxy hand 1000 is then coupled to its corresponding actuator 1010 in the touch feedback device 1004 via the controller 1020.

For example, if the proxy limb 1000 includes sensors 1008 at each fingertip, the touch feedback device 1004 preferably has actuators 1010 at locations in contact with the user's fingertips. In some embodiments, the touch feedback device 1004 may include finger clips or like devices for holding the user's real hand in the touch feedback device. Those finger clips may house the sensory actuators 1010, similar to the finger clips 504 discussed above. In other embodiments, the touch feedback device 1004 may be embodied as a glove.

Accordingly, upon touching the proxy limb 1000 with the wand 1002, the touch feedback device 1004 synchronously touches the user's real limb 1012 at the corresponding location. For example, if the user 1006 touches the wand 1002 at an index fingertip on the proxy limb 1000, the touch feedback device 1004 preferably actuates a sensory actuator 1010 in contact with the user's real index finger. The cooperation between the detection of the wand 1002 by the proxy limb 1000 and actuation of the sensory actuators 1010 in the touch feedback device is moderated by the controller. While the controller 1020 is shown in FIG. 10 as a separate control device connected between the proxy hand 1000 and the touch feedback device 1004 (in a wired or wireless manner), it should be understood that the controller may be part of the proxy hand 1000 or the feedback device 1004, with the proxy hand 1000 and feedback device 1004 directly connected to each other in a wired or wireless manner.

The user's use of the wand 1002 may be part of an interactive game. In one embodiment, the user 1006 places their real hand (on the limb the procedure is to be performed) 1012, palm up, into the touch feedback device 1004, or in a glove of the touch feedback device 1004. The touch feedback device 1004 may be hidden behind a screen 1014 affixed to a procedure table 1016, or otherwise in a visually separate field of view than the proxy hand 1000. The sensory actuators 1010 are located at each fingertip position of the feedback device 1004. The proxy or like proxy hand/arm 1000 is provided on a side of the screen 1014 that the user can see, and is posed in the same way that the hidden real hand 1012 is posed in the feedback device 1004.

In use, as noted above, when the user 1006 touches the wand 1002 to one of the fingertips of the proxy hand 1000, the wand 1002 is detected by the sensor 1008 of the proxy fingertip. In some embodiments discussed in more detail below, the wand 1002 only needs to be within a predetermined proximity of the fingertip to be sensed by the sensors 1008. The sensor 1008 then transmits a signal to the controller 1020, which identifies the location of the detection and causes actuation of the corresponding sensory actuator 1010 of the feedback device 1004. Depending on the sensors used, a pressure, proximity, or the like may also be detected and analyzed by the controller 1020 so as to cause a haptic sensation caused by the sensory actuator 1010 to more closely correspond to the actual touch by the wand 1002. The wand, in turn, may contain actuators that use the predictive nature of the proximity sensing and touch feedback to prepare the system to provide low (or negligible) latency to the feedback device 1004 that give the user 1006 the impression of fingertip compliance. Such a realistic latency can provide a more realistic perceptual illusion of game and hand naturalness to facilitate a cooperative engagement with the cognitive mechanism (the shared reality 900).

For example, the controller 1020 may cause a high intensity actuation upon a pressure sensor's detection of a high pressure touch by the wand 1002. Preferably, the controller 1020 causes actuation of the sensory actuators 1010 simultaneously (or nearly simultaneously, if realistic latency based on predictive touch is employed) with the touch of the wand 1002 to the proxy 1000. As discussed above, such simultaneous touch and feedback further strengthens the illusion and the ability of the user to cognitively neglect the real procedure hand. With respect to FIG. 9, this effect can be understood as the patent's observations aligning with expectations through the comparator 912-1 to modulate internally modeled reality 914-1 and update the shared reality 900 through the user's intent, whereas the Agent 2 devices of FIG. 10 provide information in cooperation with the user 1006 to facilitate the perceptual illusion of hand naturalness. Thus, both the user 1006 as Agent 1 and the devices of FIG. 10 as Agent 2 share agency over the cooperative task of perception of naturalness of interaction between the user and devices.

In some embodiments, the proxy hand 1000 may further include LEDs or like lights 1018 to indicate particular locations of the proxy hand 1000 for the user 1006 to touch with the wand 1002. For example, lights 1018 in each finger may be illuminated (as controlled by the controller 1020) according to a pattern in which the user is to touch each finger of the proxy hand 1000. The pattern may show only one light at a time—for example, thus waiting for a user 1006 to touch the finger corresponding to the illuminated light before illuminating another light—or may show a plurality of lights at a time—for example, requiring the user 1006 to remember a series of touches to perform after the illumination pattern is shown. Characteristics of the lights (e.g., color and intensity) may also be controlled to indicate a desired pressure of touch, speed of touch, or the like, or to help the user 1006 distinguish between the different fingers. The lights 1018 may be illuminated randomly or according to a predetermined pattern.

By performing dissociation with the above or a like game causes the user 1006 to further focus cognitive attention on the dissociation process. Further, this limits the interruption of clinical workflow because the user is in charge of establishing the cognitive disconnection while waiting for a clinician to prepare a workspace. Still further, presenting the dissociation as a game can be fun and distract the user from the impending procedure.

Referring back to FIGS. 1 and 9, the brain is highly sensitive to timing differences in the comparison of the internally modeled expected reality 114, 914-1 and the sensed reality output of valve 110 at comparator 112, 912-1. In view of this, touch events of the wand 1002 may be anticipated and compensated for by speeding up the actual sensation through feedback device 1004. In other words, unnatural delays between when the user expects to feel a touch (based on their visual perception) of the wand 1002 touching the proxy 1000, and when they actually feel touch from the sensory actuator 1010 of the feedback device 1004 can affect the illusion. Merely making the system as fast as possible to disguise inherent lag does not necessarily produce a natural response, however. Further, using another human to administer the touches on both hands is not necessarily desirable due to delays in that human's perception and the required resources of relying on other personnel.

In view of this, the controller 1020 may control the sensory actuators based on projected anticipated touch events into the future in order to compensate for electrical, mechanical, and processing lag in the illusion game. For example, because proximity sensors can recognize the presence of the wand 1002 in the proximity of the fingertip of the proxy 1000 prior to an actual touch, the sensor 1008 may signal the controller 1020 of the impending touch. The controller 1020 can then properly time its output signal actuating the appropriate sensory actuator 1010 so that the actuation begins at the time of actual touch.

By knowing the location of each sensor 1008, the controller 1020 may identify the location of the wand 1002 based on which sensor 1008 senses the presence of the wand 1002. In some embodiments, the wand 1002 may be detected by multiple sensors 1008. In these cases, the controller 1020 may predict the location of the touch by identifying the sensor 1008 with which the proximity changes at the greatest rate, thus suggesting the sensor 1008 to which the wand 1002 is moving most directly toward.

In some embodiments, velocity, acceleration, and/or motion information of the wand 1002 may also be used by the controller 1020 to predict its location by recognizing that the user 906 may naturally (at least temporarily) slow movement of the wand 1002 as the wand 1002 approaches the desired touch location, in order to increase accuracy of the touch. Thus, a sensor 1008 detecting proximity at a time corresponding to a slowing of the wand 1002 may be predictive of the location of the future touch.

In some embodiments, the controller 1020 may predict the touch location based on activation of the above-discussed LEDs 1018. In other words, because the activated lights 1018 serve as instructions for body part locations to be touched by the user 1006, the controller 1020 may assume the user will follow the instructions and thus predict the location of a future touch based on the light instructions.

In still other embodiments, the controller 1020 may analyze sensor signals from the proxy limb 1000 and/or the wand 1002 to determine both a velocity/acceleration and directionality of the wand's movement. The controller 1020 may use the directionality information to predict the touch location, and the velocity/acceleration to predict the touch time. In short, the controller may predict when, and on which finger, an actual touch on the proxy hand 1000 will occur. The controller 1020 may then cause the sensory actuator 1010 for the corresponding finger on the real hand 1012 to actuate at the predicted time of touch.

In some embodiments, a system that compensates for lag is not limited to the full system lag or no lag at all. Rather, the lag may merely be reduced, zero, negative, dithered (e.g., having a zero-mean but deliberately noisy), and the like.

The controller 1020 may also be configured to learn the behavior of the user 1006, and adjust in real-time to make more accurate and effective predictions about the timing of future touch events. For example, by considering data from accelerometers or like sensors in the wand (e.g., as recorded in a memory), the controller 1020 may determine average rate of movement of the wand. By knowing the detection proximity of sensors 1008 in the proxy hand 1000, and the speed of the wand 1002, the controller may more accurately determine when the wand will touch the proxy. In some embodiments, the controller 1020 may be or include a learned controller including, for example, a machine learning system that is continually trained by the user's wand movement. Accordingly, the controller's predictions may improve during the illusion game. These predictions by the controller 1020 may also be unique to each user.

While various features are presented above, it should be understood that the features may be used singly or in any combination thereof. Further, it should be understood that variations and modifications may occur to those skilled in the art to which the claimed examples pertain.

1. A method comprising:
operating a first sensory actuator with respect to an extremity of a user;
operating a second sensory actuator with respect to a proxy extremity, the proxy extremity corresponding to the extremity of the user; and
performing a medical procedure on the extremity of the user,
wherein operation of the first sensory actuator is visually hidden from the user and induces a non-visual sensory response in the extremity of the user, and
wherein operation of the second sensory actuator is visible to the user and does not induce the non-visual sensory response in the user.

2. The method of claim 1, wherein the extremity is a finger or hand.

3. The method of claim 1, wherein the extremity is a toe or foot.

4. The method of claim 1, the first sensory actuator and the second sensory actuator forming an sensory actuator pair and the method comprising operating a plurality of sensory actuator pairs,
wherein each sensory actuator pair corresponds to a different extremity of the user, and
wherein the sensory actuators of each sensory actuator pair are operated simultaneously.

5. The method of claim 1, the first sensory actuator and the second sensory actuator forming an sensory actuator pair and the method comprising operating a plurality of sensory actuator pairs,
wherein each sensory actuator pair corresponds to a different extremity of the user, and
wherein the sensory actuators of each sensory actuator pair are operated asynchronously.

6. The method of claim 1, the first sensory actuator and the second sensory actuator are operated until the user at least partially disembodies the extremity.

7. The method of claim 1,
wherein the first sensory actuator is not visible to the user during operation of the first and second sensory actuators, and
wherein the second sensory actuator is visible to the user during operation of the first and second sensory actuators.

8. The method of claim 1, wherein the first sensory actuator is a rack and pinion actuator, and operating the first sensory actuator comprises controlling a servo motor of the rack and pinion.

9. A system comprising:
a lower frame;
an upper frame rotatably attached to the lower frame;
a real hand plate attached to the lower frame and configured to hold a hand of a user;
a proxy hand plate attached to the upper frame; and
a proxy hand held on the proxy hand plate,
wherein the real hand plate comprises a plurality of first sensory actuators and the proxy hand plate comprises a plurality of second sensory actuators, and
wherein each one of the second sensory actuators is at a location of the proxy hand corresponding to a location of one of the first sensory actuators of the user's hand.

10. The system of claim 9, wherein each of the first sensory actuators is at a different finger of the user's hand, and each of the second sensory actuators is at a different finger of the proxy hand.

11. The system of claim 10, wherein the real hand plate and the proxy hand plate each comprise a plurality of finger clamps attached to the plate, each of the plurality of finger plates housing a different one of the first and second sensory actuators and being configured to hold a finger of the user's hand or the proxy hand.

12. The system of claim 10, wherein each of the first and second sensory actuators is a rack and pinion actuator comprising a servo configured to rotate the pinion.

13. The system of claim 9, further comprising a processor configured to:
simultaneously operate one of the first sensory actuators and the one of the second sensory actuators at the corresponding location of the proxy hand.

14. The system of claim 9, further comprising a processor configured to:
operate one of the first sensory actuators and the one of the second sensory actuators at the corresponding location of the proxy hand at different times, or
simultaneously operate one of the first sensory actuators at a location of the hand of the user and one of the second sensory actuators at a different location of the proxy hand.

15. The system of claim 11, wherein an angle between the lower frame and the upper frame is greater than zero degrees such that when the sensory actuators are operated, the user's hand and the first sensory actuators are not visible to the user, and the proxy hand and the second sensory actuators are visible to the user.

16. The system of claim 9, wherein the proxy hand is a digital representation of the hand of the user on a display, the display being held on the proxy hand plate.

17. A system comprising:
a touch feedback device comprising a plurality of first sensory actuators and configured to hold an extremity of a user;
a proxy extremity; and
a processor configured to operate one of the first sensory actuators a location of the extremity of the user corresponding to an identified location of the proxy extremity,
wherein the extremity of the user and the proxy extremity are in a visually separated fields of view, and
wherein the plurality of first sensory actuators are configured to induce a sensory response of the user when operated.

18. The system of claim 17, further comprising:
a wand,
wherein the processor is configured to determine the identified location of the proxy extremity based on a proximity or touch of the wand to the proxy extremity.

19. The system of claim 17, wherein the processor is further configured to:
predict the identified location of the proxy extremity based on a movement of the wand or the proximity of the wand to the proxy extremity; and
operate the one of the first sensory actuators prior to the wand touching the proxy extremity.

20. The system of claim 17, wherein the proxy extremity is a digital representation of the extremity of the user.
